(19) United States
(12) Patent Application Publication
(10) Pub. No.: US 2010/0136608 A1
(43) Pub. Date: Jun. 3, 2010

(54) Multiple Filter Array Assay

(75) Inventors: David L. PUTNAM, Sammamish, WA (US); Jason A. Putnam, Sammamish, WA (US)

Correspondence Address: MORGAN LEWIS & BOCKIUS LLP, 1111 PENNSYLVANIA AVENUE NW, WASHINGTON, DC, 20004, US

(73) Assignee: Photonic Biosystems, Inc., Sammamish, WA (US)

(21) Appl. No.: 12/582,447

(22) Filed: Oct. 20, 2009

Related U.S. Application Data
(60) Provisional application No. 61/106,623, filed on Oct. 20, 2008.

(51) Int. Cl. C12Q 1/04; G01N 1/34; G01N 33/00

(52) U.S. Cl. 435/34; 436/178; 422/68.1

(57) ABSTRACT

The present invention relates to a method and device for the assay and determination of the concentration of organisms or other filterable constituents of a fluid test sample. The method includes the steps of exposing, processing, and analyzing a liquefied sample in a filter tray apparatus. Each filter element (FE) filters a volume of liquid from a common fluid test sample. Excess unfiltered liquid and filtrate is removed from the filter tray apparatus. Each filter element contained in the filter tray apparatus is exposed to culture medium and/or appropriate testing reagents introduced to the apparatus to generate an indicator of the filterable constituent. Final examination is conducted on the filter tray apparatus for the indication of the presence and/or absence of filtered constituents in the multiple filter elements. The presence/absence information defines the concentration of the filterable constituent in the fluid test sample.

### CROSS-REFERENCE TO RELATED APPLICATIONS

This application claims the benefit of U.S. Provisional Application No. 61/106,623, which was filed on Oct. 20, 2008, hereby incorporated by reference.

### STATEMENT REGARDING FEDERALLY SPONSORED RESEARCH

The U.S. Government owns rights in the present invention pursuant to contract number W81XWH-04-C-0149 from the United States Department of Defense through the US Army Medical Research Acquisition Activity.

### BACKGROUND OF THE INVENTION

Tests to detect and enumerate or determinate concentrations of microorganisms are very important in water, beverage, food, medical and pharmaceutical, and many other industries. The needs to enumerate the organisms are diverse, ranging in size and in nature, e.g., 1 mL to 100 mL volume liquid samples, encompassing liquids to solid foods and meat-cutter equipment.

For enumeration, in particular, a variety of classic microbiological tests are used, each having its own set of advantages, and limitations of suitable applications. For instance, solidified agar pour-plate and spread-plate tests methods are typically limited to analysis of several milliliters of sample cultured in a Petri dish. The membrane-filtration (MF) technique has an advantage of collecting a small number of organisms from large volumes of liquid, when the test sample is a fluid and filterable. The MF process involves first harvesting the fluid in a filtration system, then transferring the filter to a culture vessel where it is exposed to media. The vessels are incubated at a certain temperature for a specified time allowing for organisms to grow into visible colonies on the membrane.

Membrane-filtration has several common problems. Some sample materials are not readily filtered because of particulate content that clogs the pores of the filter matrix. It has to be performed carefully in a clean environment to avoid contamination and can be technically tedious to execute.

Identification and enumeration of the grown colonies on a membrane can require skilled personnel and careful microscopic examination to obtain a reliable colony count, especially if there are many colonies, colonies of different types, and particulate matter to visually differentiate.

Problems with the membrane-filtration approach can be avoided by using the multiple-tube method, which involves dilutions of the sample in broth cultures. The technique referred to as the Most Probable Number or MPN method requires that a volume of sample be diluted several times over a dilution range, with several replicate tubes prepared of each dilution set. Culture media is added to the tubes and they are incubated for an appropriate time to produce a visible indication of the presence of the organism of interest. The tubes exhibiting a positive outgrowth are counted. Based upon the dilution level and number of tubes that go positive, the most probable number of organisms that were present in the original undiluted samples can be determined A formula for the derivation of the MPN based prediction of the cell concentration is published.

The MPN technique using broth-based tube cultures is advantageous for samples containing too much particulate for membrane-filtration. Since readout is based on ascertaining simply presence versus absence of growth in individual tubes, it avoids the technical challenge of scoring individual colonies on a plate or membrane-filter culture. However, the MPN takes more time, materials, and effort to set up the multiple tube dilution cultures.

Another method replaces the tubes with carrier trays having multiple compartments that hold aliquots of the test solution. The test sample mixed with media is divided within the devices into portions that are distributed to individual compartments or wells to hold defined quantities of fluid. The compartments of fluid are incubated until organism outgrowth can be scored. Like the MPN, based on the compartments exhibiting positive indication of organisms, a prediction is then made of their original concentration in the parent sample.

The compartmentalized tray offers a convenience over preparing MPN tubes, but it has shortcomings for analysis of large-volume samples bearing relatively few organisms. The culture of a relatively large amount of fluid in a plurality of wells makes the devices to be incubated much larger than a classic membrane-filter or a Petri dish. Additionally, since organisms are grown in the parent sample fluid, if its color or constituents interfere with readout scoring of results, and if there are inhibitory agents or toxics contaminating the sample that interfere with organisms growth (“matrix effects”), then test errors and inaccuracies will occur.

The present invention provides an improvement to current enumeration techniques by enabling a test method that is easy to prepare, has a small format culture device, allows large fluid volumes to be assayed, employs a non-technical readout based on presence/absence scoring, and removes the parent sample from the culture condition in order to avoid complications of matrix-effect interferences. It combines novel implementation with features of the current Membrane-filtration, MPN, and compartmentalized tray methods.

### SUMMARY OF THE INVENTION

The present invention provides for a simple assay method and device to enumerate organisms and/or determine the concentration of filterable entities in a fluid sample. It uses a filtration-based approach to sample preparation that distributes one common sample between a plurality of non-communicating separate filter elements (FE). The test fluid introduced and exposed to the inlet side of the filter elements is filtered through the filter elements, each element filtering a defined portion of fluid from the sample pool. Each filter-element portion can be the same volume, or of different volumes in order to provide a dilution effect. The filter element captures the entity to be assayed and retains it while it is subsequently exposed to preferred analytical procedures.

The analytical process may be such that a positive measure of the presence of the entity of analysis, or its absence, can be ascertained without further procedures. Alternatively, the invention provides for additional procedures that preferably involve rinse agents and reagents. These include procedures that provide indicating-means which serve to identify the presence of the filtered entity or entities being the objective of analysis. One or more readouts are preferably conducted over a period of time to ascertain or measure the presence or absence of the indicator product engendered at the site of each filter element. On the basis of the number, pattern, or distribution of positive filter elements that exhibit the indicator, versus negative filter elements, a determination is made of the concentration of the filterable assay entity present in the parent sample at the onset of the assay.

The inventive method and device in preferred embodiments is applied to the assay of organisms to detect those that grow and replicate and thus require time for their culture, and to the assay of entities the presence of which involves use of reagents that require time for an indicator-generating means to engender identifiable indicator. In such cases, the inventive method additionally provides for steps of: exposing each filter element to an appropriate culture media or reagent(s), enabling the generation of an indicator that identifies the presence of the entity of analysis; preferably covering the filter-elements to protect from drying or contamination during the period of incubation required for the development of the indicator; incubating the filter-elements for an appropriate time under appropriate temperature or other conditions preferred for generation of the indicator; analyzing the filter-elements for the presence of indicator preferably more than once over a period of time. The concentration of the filterable assay entity present in the parent sample at the onset of the assay is determined based on the information contained in the number, pattern, and/or distribution of positive filter elements that exhibit the indicator.

Additionally, the inventive method provides for preferably assessing the presence of indicator in the filter apparatus repetitively over a period of time in order to facilitate the determination of the concentration of entity in the parent sample. The time when particular wells exhibit positive evidence of indicator and which do not, in conjunction with the knowledge of the volume of sample filtered by the respective filter elements, is useful information that provides a second basis of establishing the concentration of assayed entity. Preferably the repetitive measures of filter elements includes a measurement at a time before any of the indicating means used for the analysis have completed their assay reaction process and before all filter-elements in the filter apparatus have turned positive.

The inventive method and device in a preferred approach provides for media or other reagent(s) to be presented to the filter-element in a convenient manner by administering via a component of the device to which they are pre-applied. When contacted with the filter-element, media or reagent is delivered to or diffuses to it. This includes the implementation of dry or lyophilized media or reagent, which upon exposure wetted filter-elements will be hydrated and thereby be delivered to the filter-element.

The inventive method and device further provides for use with implementation of a volume-limiting filtrate collection apparatus. This device has a plurality of separate fluid collection chambers each providing a fluid path to mate and form a seal with the fluid path of its corresponding FE on the outlet side of the filter tray apparatus. The collection chambers provide the receipt of defined and controllable amounts of filtered fluid, which limits the volume of fluid each FE is allowed to filter. Each collection-chamber acts independently, to acquire its specified volume of filtrate, and upon completion of collecting that volume, separately terminates the transfer of fluid via its respective FE. The collection apparatus thereby prevents each FE in the filtration apparatus from filtering more than the prescribed volume of fluid from the sample pool presented to the FE.

To terminate the fluid filtration, each collection chamber is an enclosure that provides for a vent or valve mechanism capable of stopping fluid entry upon receipt of a prescribed volume of filtrate. The vent approach provides preferably for a porous matrix or valve that enables passage of air such that only a predefined volume of air can exit the chamber and a predefined volume of fluid can enter the chamber. The vent is incorporated into the collection chamber in a position such that at the predefined volume the fluid level blocks the vent and no more fluid can enter the collection chamber. The porous matrix is preferably a material that does not allow passage of aqueous fluid.

The inventive method and device provides for the sample preparation by filtration to be performed using negative pressure based methods and not requiring of necessity positive pressure fluid delivery means such as gravity feeding or pump-based fluid paths.

It is to be understood that both the foregoing general description and the following detailed description are exemplary and explanatory and are intended to provide further explanation of the invention as claimed.

### DESCRIPTION OF THE DRAWINGS

FIG. 1 is a cross section side view illustrating an exemplary filter tray apparatus with inlet and outlet sample processing accessories. As shown, the filter tray apparatus (109) is sandwiched between the inlet sample holding chamber (101) and the outlet chamber (105). When assembled, the three parts become intimately sealed. Fluid samples are introduced to the inlet sample holding chamber through various means including suction pick up, or simple pouring of the sample. Ports between the individual FE chambers allow the fluid sample to fill and distribute precise volumes to each FE. Excess fluid escapes through the air and fluid outlet port (102) in the outer wall of the inlet sample holding chamber (101). Once all excess fluid is removed and each individual FE chamber is precisely filled, a vacuum is applied to the outlet chamber and the fluid samples are filtered through the pre-filter (110), hydrophilic filter element (104), and the post-filter (106). Filtrate is evacuated through the air and fluid outlet port (103) to complete the filtration process. Additional drawing elements: Hydrophobic Vent (107); Air Vent (108); Air and Fluid Inlet Port (111); Filter-Element Chamber Ports (112).

FIG. 2 is a cross section side view illustrating an exemplary filter tray apparatus with alternative inlet and outlet sample processing accessories. As shown, the filter tray apparatus (203) is sandwiched between the inlet chamber (201) and filtrate-collection compartment (205). When assembled, the three parts become intimately sealed. Fluid samples are introduced to the inlet chamber (201) through suction by applying a vacuum to the hydrophobic vent (210) on the inlet chamber. Once the inlet chamber contains adequate fluid sample, filtration can begin by applying vacuum to the air outlet port (204). Negative pressure is consequently created in each of the individual porous vented collection chambers (206) and fluid sample is pulled through the pre-filters (209), hydrophilic filter elements (212), and post-filters (208). Once the porous vented collection chambers have filled, no more water can be drawn through the filter tray apparatus (203) and the filtration process is complete. Additional drawing elements: Fluid Inlet Port (202); Hydrophobic Vents (207); Hydrophobic Vent (210); Air Vent Out (211).

FIG. 3 is a cross section side view illustrating an exemplary filter tray apparatus with subsequent processing accessories. As shown, the filter tray apparatus (308) is sandwiched between the inlet chamber (301) and outlet chamber (306). When assembled, the three parts become intimately sealed. In this mode, rinses and reagents can be exposed to the hydrophilic filter element (304). Fluids are drawn into the inlet chamber (301) by applying vacuum to the inlet chamber vent (310). Vacuum is then applied to the outlet chamber port (303) pulling reagent and rinse fluid through the pre-filter (311), FE (304), and post-filter (305). Excess fluids can be left in the inlet chamber (301) or they can be evacuated by removing the fluid source while continuing to apply vacuum to the outlet chamber. At this point, the assembly can then be put into assay or disassembled for further processing of the filter tray apparatus (308). Additional drawing elements: Fluid Inlet Port (302); Air Vent (307); Hydrophobic Vent (309).

FIG. 4 is a cross section side view illustrating an exemplary filter tray apparatus with final assay accessories. As shown, the filter tray apparatus (405) is sandwiched between a top cover (401) and bottom cover (402). When assembled, the three parts do not necessarily become intimately sealed. Depicted on the bottom cover, though also applicable to the top cover, are attached media/reagent pads (403). These pads contain dehydrated or hydrated materials required for the particular assay of interest which diffuses into the hydrophilic filter element (407) through the fluid remaining in the Pre-filter (406), FE (407), and post-filter (404) after processing. Each pad communicates only with its corresponding FE and no other in the filter tray apparatus. Once assembled, the device is put into assay for subsequent analysis.

The accompanying drawings, which are included to provide a further understanding of the invention and are incorporated in and constitute a part of this specification, illustrate embodiments of the invention and together with the description serve to explain the principles of the invention.

### DETAILED DESCRIPTION OF THE INVENTION

The goals this invention accomplishes are:
1) Enable an in vitro quantitative analysis of the concentration of an entity in a fluid sample.
2) Derive an assay method that has easy and faster setup and execution than existing techniques; for instance, reducing the complexity of the multi-tube MPN test approach.
3) Analyze large volume fluid samples, e.g., 100 cc of water, as well small volumes.
4) Analyze food or beverage samples that are difficult to filter by conventional membrane filtration.
5) Provide simple readout of the analytical procedure and reduce the technical skill required
6) Provide for analyses that can rely on a Presence/Absence readout based approach to make quantitative determinations of the concentration of an entity in a fluid sample.
7) Reduce the equipment and resources required to perform the assay procedures.
8) Increase the range of environments in which the assay can be performed by reducing needs for clean facilities and aseptic conditions to prevent contamination of samples under assay.
9) Reduce the amount of materials, disposables, and equipment required for fluid handling in preparing the assay sample, thereby reducing the materials contaminated by sample handling.
10) Allow for analyses of constituents of a fluid sample by providing for the fluid to be removed in order to reduce the sample volume and avoid analytical complications associated with the presence of other interfering substances also carried in the fluid.

The scope of application is a filterable fluid sample and the in vitro analysis of test entities that can be removed from the fluid by filtration. The test entity can be molecules in a solution, particulate matter, or biological material, which includes microorganisms, cells and higher multi-cell organisms. Removal of the test entity by filtration processes includes any mode of separation whereby the entity can be captured, bound, and/or entrained by the filter element such that the test entity is selectively retained or immobilized and thereby concentrated from the fluid passed through the filter element.

The basis for the filter retention of the test entity can be chemical, biochemical, or physical properties of the entity, such as size or surface charge. Fluid can be moved past the filter-element, preferably by negative-pressure or vacuum based approach applied to the outlet, discharge, or post-processed side of the filter apparatus so as to draw or pull fluid through from the inlet side. Alternatively, positive pressure can be employed to push fluid through the filter-element from the upstream, incoming feed, or inlet side.

The following provides a detailed description of device embodiments and exemplary methods of use.

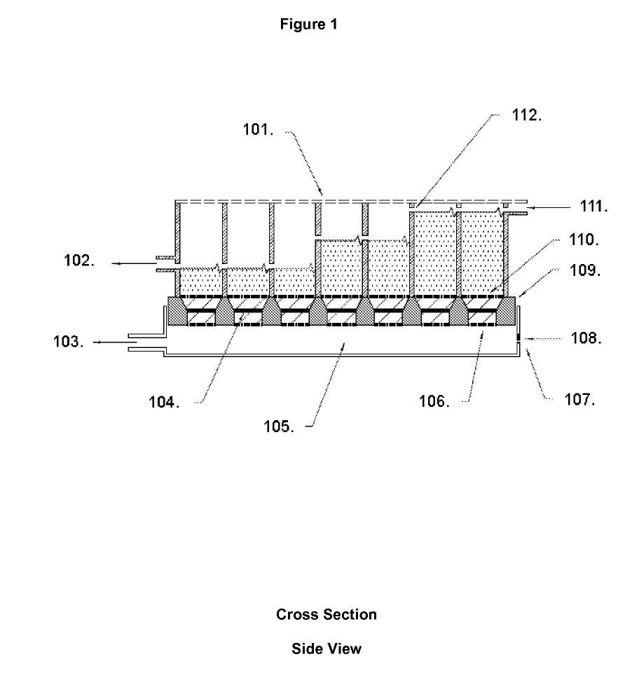

The filter apparatus consists of a filter tray apparatus having multiple (e.g., 3-200) holes or separate fluid pathways through it. Each non-communicating pathway is covered with a filter element (FE), sealed and bonded such that the fluid path can only be through the FE and not around it. Reference FIG. 1. The FE and pathways can all be of the same size with each FE designed to filter the same volume of sample fluid (e.g., 1-10 milliliters). However, the preferable implementation is to incorporate FEs that filter different quantities of fluid, preferably covering a range, such as 0.1, 1, 10, and 100 mL. This is to accomplish the same result as a test of dilutions of the sample, with replicates at each dilution level, simulating an MPN type test. The multi-volume, or dilution type approach, affords the determination of the concentration of an entity in a fluid over a wider range, and requires fewer FEs as opposed to equally sized test aliquots.

In regard to capturing particulate matter and microorganisms from aqueous solutions, the filter elements are made of size-exclusive, porous, hydrophilic membranes or matrices. Once wetted, these membranes generally will not allow air to pass through the pores. As known in the art, typical filter media have different pore sizes, retention capabilities, and properties. For filtration of microorganisms, typical examples are filter membranes with pores of 0.2 to 0.45 microns, made of materials such as cellulose acetate, mixed cellulose esters, polyether sulfone, polyvinyl difluoride, or nylon.

The test fluid is introduced to the inlet side of the tray so that the FEs are exposed to it. In one manner of use, to harvest a sample from either a beaker or a lake, the inlet face of the FE tray is immersed in the sample source, putting the FE in direct contact with the sample source pool. Alternatively, a sample of the test fluid is delivered to the filter apparatus. In this mode, the filter apparatus provides for an inlet chamber over the multiple FEs, which serves as a reservoir able to hold minimally the volume of fluid to be filtered collectively through the FEs.

In one example, reference FIG. 1, the inlet sample-holding chamber (101) provides a perimeter wall of sufficient height around the filter tray apparatus (109) to create a reservoir containing the required sample volume. The sample fluid is introduced into the chamber, from which each FE is able to withdraw and filter its portion. The perimeter wall can be a removable part in order to reduce the height of the filter apparatus subsequent to the sample filtration.

In preferred embodiments the fluid flow to the inlet chamber is accomplished using vacuum to pull or aspirate fluid into the inlet chamber from a sample-fluid vessel or source. An exemplary design embodies an inlet chamber (101) that when attached to the FE tray (109) constitutes an enclosure. The inlet chamber (101) in FIG. 1 contains a lid or cover mating with the chamber walls, an outlet port (102), and a fluid inlet port (111). Vacuum applied to the inlet-chamber outlet port (102) creates suction in the chamber which draws fluid from the sample vessel through the fluid inlet port (111) and into the inlet chamber. The chamber is filled to adequately expose the FE to fluid. The outlet vent (102) can be covered with a hydrophobic micro-porous membrane that passes air but not liquid. Such a vent prevents sample fluid from being further sucked out of the vent and it also prevents contamination from sources external to the filter apparatus from entering the inlet chamber via the vent, e.g., bacteria.

Various receiving chambers and fluid distribution approaches can be conceived for introducing incoming-fluid in keeping with the inventive concept. The precepts are: enable distribution of fluid from a common sample source to multiple FEs; enable each individual FE to independently filter a specific and precise portion of the sample fluid, which may be the same or different volumes between FEs; enable each compartment, once filled, to be non-communicative for the filtration process.

With the FEs exposed to sample fluid, the next step is to filter the fluid through each FE. A preferred embodiment is to draw the fluid through the FEs using vacuum applied to a chamber on the outlet or discharge side of the filters. For this implementation an outlet chamber enclosure (105) in FIG. 1, which can be removable, mates with the FE tray (109). A vacuum source attaches to the outlet chamber at its air/fluid outlet port (103). Negative pressure in the outlet chamber is thereby commonly applied at each FE causing fluid in the inlet chamber (101) to be pulled and filtered through the FEs into the outlet chamber.

In order to control and limit the amount of sample fluid filtered by each FE to a defined volume, a basic approach is to have preformed compartments, e.g., open top recesses or wells, incorporated into the inlet side of the FE tray to hold defined aliquots of fluid. Such approach is illustrated in FIG. 1, showing seven such compartments filled with fluid. Each FE has its own separate compartment. When fluid is introduced to the inlet chamber (101), these compartments serve to partition the common sample fluid between the individual FE according to the ascribed volume to be filtered. Each compartment is flooded and/or loaded with its aliquot. Any excess fluid is removed. The individual sample wells are arranged such that once the fluid has been partitioned to each compartment, they are non-communicative with the original common sample or the other individual sample wells. The filter apparatus can be held or oriented, such that no additional sample fluid is able to get into any compartment nor the sample wells able to communicate said sample fluid while the filtration is in progress.

This approach, with the provision that the common fluid be distributed to fill each compartment with only the prescribed amount, and no more, is simplistic and practical. Fluid dispensing and metering equipment can be used to deliver defined quantities of fluid to each FE, or respective inlet compartment.

An alternative preferred approach is to define and limit the volume filtered by each FE from the outlet side of the apparatus on the basis of the fluid passed through the filter (i.e., filtrate), stopping the filtration when the specified volume has been processed. There are distinct advantages to this approach. It addresses concern about sample cross-contamination. As a collection apparatus operating on the outlet side of the filter, all fluid it contacts comes into it through the FE and has been filtered. Additionally, the FE does not contact material that is on the downstream side. Therefore, there is minimal or negligible risk of the FE assay becoming contaminated by the collection apparatus or cross-contaminated when it has been previously used to processes other samples. Consequently, the collection apparatus can be used repetitively. It does not have to be a part of a disposable device. Moreover, the size of the collection apparatus can be relatively large to accommodate the assay volume; but that is immaterial to the size of the filter apparatus. To advantage, the filter apparatus can remain relatively small and compact thereby less expensive and more convenient for performing the subsequent assay procedures.

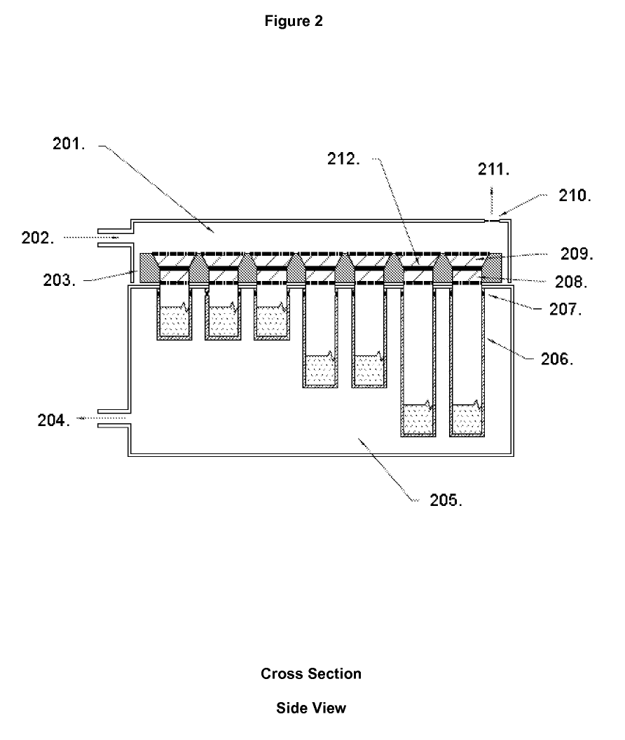

In the preferred case of limiting fluid filtration volumes at the outlet side, another advantage is that the size and amount of material required for an inlet chamber is reduced. Referencing FIG. 2, the inlet chamber (201) is made to have a small volume, with no intention that it hold the entire amount of sample fluid to be filter processed. It need only be large enough to ensure presentation of fluid to all FEs (one FE (212) identified in FIG. 2) during the filtration. To accomplish this, it provides for a fluid path to an external sample reservoir from which additional fluid is withdrawn to replenish what is removed from the inlet chamber during filtration. The path can simply be a tube to a sample bottle. Fluid flow to the inlet chamber can be achieved using a gravity feed or pump assistance; but a preferred method is to utilize vacuum applied to the hydrophobic vent (210) to aspirate fluid into the inlet chamber to keep it filled with fluid.

The preferred method and device for controlling the maximum volume of fluid filtered by each FE from the outlet side provides for such an apparatus consisting of a volume-limiting filtrate collection compartment (025), which contains and houses commonly multiple collection-chambers (206) (one identified in FIG. 2), a separate one for each FE (reference FIG. 2). Every collection chamber mates and forms a seal with the fluid path on the outlet side of its corresponding FE in the filter tray apparatus (203). As the FE filtration step is executed, the collection chambers receive and hold the fluid from the FE, i.e., the filtrate. Every collection-chamber independently acquires its specified volume of filtrate. Upon collecting that volume, each collection chamber terminates its FE transfer of fluid from the inlet chamber (201), i.e., stops filtration. Thus the FE is prevented from filtering more than its designed volume of fluid from the sample pool presented to it.

A preferred embodiment to terminate fluid filtration, consists of individual collection chambers each having a vent or valve mechanism capable of stopping fluid entry upon receipt of the prescribed volume of filtrate from the FE. The vent allows negative pressure to be produced in each collection chamber by a common vacuum applied to the compartment housing them.

The vent approach provides preferably for a porous matrix or valve that enables air to pass in and out of the collection chamber through the vent, but not fluid. A vent is incorporated into a wall or aspect of the collection chamber (one example shown in FIG. 2 with vent (207) and collection chamber (206)). Filtered fluid enters the chamber as long as air can move or be displaced out of the chamber enclosure; namely, the air volume that can be displaced is made to be the same as the predefined volume of fluid to be collected. This is implemented by placing the vent in the chamber such that the acquisition of the specified chamber fluid volume covers the vent, which blocks airflow out. Since air is blocked and fluid cannot escape by the vent, no more filtered fluid from the FE can enter the collection chamber.

A suitable vent material is a porous hydrophobic matrix that passes air but not fluid, such as a porous PTFE membrane as previously described. The vent enables fluid (filtrate) to be drawn into the enclosure and not allow escape. To be noted, in terms of utilization, the vent does not necessarily have to be a small aspect of the enclosure. In the case of the outlet fluid collection-chambers, the vent may be a substantial or major portion of the chamber forming wall. For instance, a chamber in the configuration of a cylinder can have all or a majority of the cylinder walls be made of vent material.

The vented or gas-permeable multiple collection chambers (206) in FIG. 2, are a preferred embodiment and are collectively contained within an encompassing larger compartment (205) to which vacuum is applied at port (204) for air removal. Thereby, the compartment under vacuum produces a negative-pressure within each of the gas permeable collection chambers. That condition draws fluid through the FE from the inlet chamber (201) containing the test fluid sample. The vacuum system for the compartment surrounding the collection chambers can also be simultaneously shared with the inlet chamber, used as described previously, to aspirate fluid and keep the inlet chamber filled until the filtration step is completed.

There are other approaches that can be utilized to achieve the same functional objectives. For example, the vent mechanism could operate like a flap which floats, such that a rising fluid level pushes it against a vent hole to block it. A ball-float, or more complex type carburetor float mechanism, or one-way valve can also be devised to terminate fluid flow at a certain point.

Other examples of approaches to limiting the volume of fluid filtered by each FE include the use of a piston and cylinder that connect to the outlet of the FE. Like a syringe, retracting the piston in the barrel creates negative pressure, draws fluid in and defines a specified volume of filtrate to collect. Another example is collection-chambers consisting of collapsed or expandable flexible plastic reservoirs for each FE. Fluid drawn into the reservoir, either by vacuum applied on the outlet side of the filter apparatus, or from pressure on the inlet side, fills the reservoir to capacity. With full extension of the flexible reservoir, the flow of fluid stops.

Alternative exemplary methods for controlling a fluid volume filter by an FE are based on the size or active filtration-area of the FE, the size of its pores, or the types of material used to make the FE. Such approaches modify/regulate the filtered volume based on differences in filtration rate, i.e., limiting the volume filtered as a function of the filtration rate and the filtration-time allowed. For example, doubling the diameter of an FE membrane filter disk, producing a four-fold increase in active filter surface area, proportionally increases the filtration rate by four-fold. Similarly, doubling the size of the pores in a filter membrane designed for microbial capture, from 0.2 microns to 0.45, roughly doubles the filtration rate. Some filter materials of the same pore size have intrinsic differences in flow rates under a given condition, which can also be used to advantage for achieving different filtration volumes. However, it is preferable in many cases to have a limiting-volume technique, such as in the other previously described model approaches, that is based on the absolute volume filtered rather than on differences in flow rates.

### Examples of Assay Procedures

An example of the execution of an assay to determine the concentration of E. coli and Coliform bacteria in a sample of drinking water is as follows. The required volume of the test sample per EPA guidelines is 100 mL. In a preferred embodiment, the filtration apparatus consists of a multi-filter tray apparatus having a four log range of test volumes with the following distribution of the number of FEs at each level: 10 FEs@0.1 mL, 9FEs@1 mL, 9 FEs@10 mL, and 1 FE at 100 mL. (This scenario actually provides for a duplicate assay, with two test volumes, 200 mL total sample tested.) The FEs for filtering could all be the same size; but in this example the FE are different diameter circular disks: 6 mm for the 0.1 and 1 mL aliquots; 13 mm for the 10 mL; and 18 mm for the single 100 mL FE. The FE are 0.45 micron pore polyether sulfone membranes bonded to holes in the tray apparatus.

To remove large particulate in the fluid sample and alleviate plugging of the 0.45 micron filter, a pre-filter pad, made of cellulose fibers forming a matrix of much larger porosity, can optionally be implemented as well. It is positioned on top or immediately upstream of the main FE bacterial retentive filter. A preferred embodiment incorporates a similar matrix pad beneath the FEs, on the downstream or outlet side, to provide support for the FE and to absorb and retain fluids.

The filter tray apparatus on the inlet fluid side has attached to it a removable plastic lid, or cover, that forms an inlet chamber, with a gas-permeable vent. Vacuum applied at the vent pulls fluid from a large-volume sample vessel through an inlet tube attached to the fluid inlet port on the inlet chamber.

On the outlet side of the filter tray apparatus is attached a removable volume-limiting filtrate collection compartment. It contains multiple collection chambers. The collection chambers, one for each FE, have gas permeable fluid barrier membrane vents that permit only the prescribed volume of filtrate to be filtered by the respective FEs. Vacuum is applied to a port on the filtrate collection compartment.

The filter tray apparatus, with attached inlet chamber and filtrate-collection compartment, is operated under vacuum causing the sample fluid to be filtered until all collection chambers have acquired the predefined volume of filtrate.

In the simplest form of assay, once filtration is completed, the FE is examined for the presence of filter-harvested material and the identification of the entity of assay. For assays requiring more analytical procedures, the following are preferred approaches.

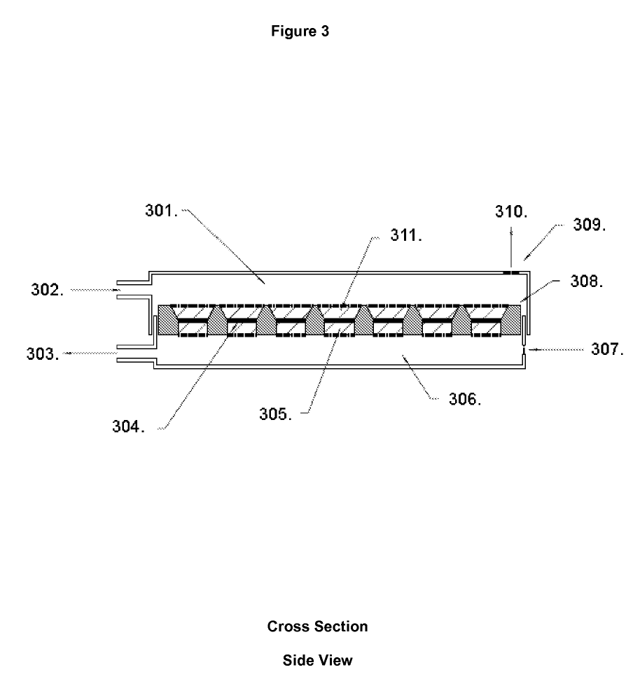

To administer another fluid agent via the inlet chamber per the model depicted in FIG. 1, e.g., for rinsing the FEs and filtered material after the filtration step, residual sample test fluid in the inlet chamber, if any, is first drained out or removed. Then rinse fluid is introduced into the inlet chamber and filtered through, by vacuum applied at the outlet chamber. A preferred approach is to introduce rinse fluid to the inlet chamber by aspirating from a rinse vessel, under vacuum, in the manner previously described for aspirating sample fluid into an inlet chamber. An alternative is to remove the sample-holding chamber (101) in FIG. 1, from the filter tray apparatus and replace it with a simpler cover forming a smaller volume inlet chamber, (301) in FIG. 3. Rinse fluid is preferably aspirated into the inlet chamber, filling it from a rinse vessel using vacuum applied to the inlet chamber vent. Vacuum applied to the outlet chamber draws the rinse agent past the FEs and into the outlet chamber from which it is discharged via the vacuum port. Preferably the inlet chamber vent is covered with a gas-permeable, fluid impervious microporous membrane.

To administer another fluid agent via the inlet chamber (201) per the model depicted in FIG. 2, there are several alternatives.

One alternative is to have the previously described filtration collection compartment designed with special collection chambers allowing both receipt of a defined volume of filtrate in the filtration step, and also provide for additional fluid to be collected in subsequent procedures such as rinsing. One approach to this, with vented chambers, is to implement the vent such that changing the orientation of the compartment, e.g., rotating 90 degrees, causes the initial sample filtrate to no longer block the vent. So vacuum in the compartment will re-enable suction in the collection chambers, which reinitiates filtration and thereby additional flow of rinse fluid past the FE.

Another option, if specific rinse or reagent volumes are necessary, is to use a controlled filtrate volume compartment with collection chambers, such as previously described.

A third scenario is to replace the filtrate-collection compartment with an outlet chamber (306) having no collection chambers. Reference FIG. 3. Vacuum is applied to a vent (309) on the inlet chamber aspirating rinse fluid into the inlet chamber (301). Vacuum applied to the fluid outlet port (303) of the outlet chamber draws the rinse agent past the FE and into the outlet chamber from which it is discharged via the outlet port.

To terminate a fluid administration, several approaches are useful: stop the vacuum at the outlet port; stop the flow in the inlet port fluid pathway; remove the fluid source from the inlet port fluid pathway; and/or allow air to be pulled into the inlet chamber as fluid is drawn out of the inlet chamber into the outlet chamber.

Using the same approaches described for a rinse fluid, other reagents including media can be introduced into the inlet chamber, exposed to the FEs, and drawn past the FEs into the outlet chamber. It has been found that this procedure is quite efficient and if the volume of the inlet chamber is kept small only small then amounts of reagents are required, e.g., 1 mL.

The inventive method and device in preferred embodiments is applied to the assay of organisms to detect those that will grow and replicate, or to detect the presence of entities which involve reagents that require time for an indicator-generating means to engender identifiable indicator.

In both such cases, the situation involves time and/or an incubation condition needed for an assay reaction to develop. The inventive method, for those cases, additionally provides steps of: exposing each filter element to an appropriate culture media or reagent(s), enabling the generation of an indicator that identifies the presence of the entity under analysis; preferably covering the filter-elements to protect from drying or contamination during the period of incubation required for the development of the indicator; incubating the filter-elements for an appropriate time under appropriate temperature or other conditions preferred for generation of the indicator; analyzing the filter-elements for the presence of the indicator preferably more than once over a period of time. Thereby, based on the number, pattern, and/or distribution of positive filter elements that exhibit the indicator, the concentration of the filterable assay entity present in the parent sample at the onset of the assay is determined

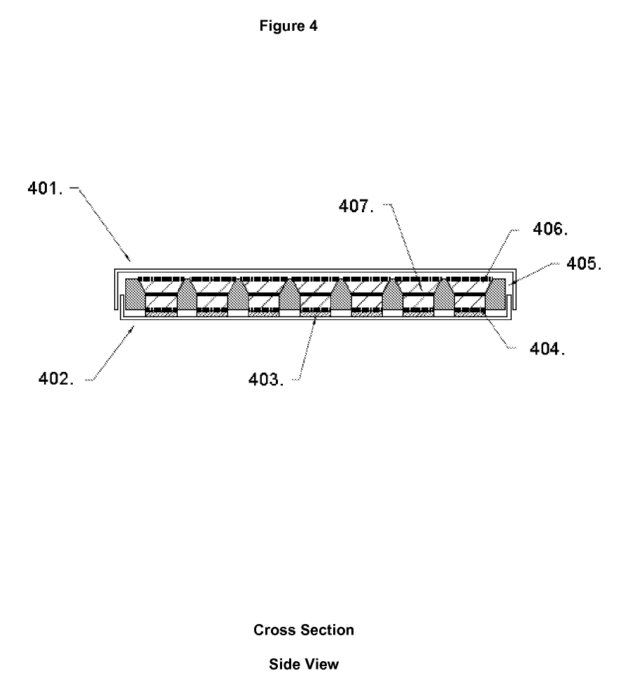

With respect to delivery of reagent or media to the FEs, a preferred approach is to provide for the reagent pre-applied to a component of the filter assay apparatus, for instance a bottom cover (402) (FIG. 4). Such a cover is needed in any case to enclose the apparatus for protection, avoid contamination, and prevent drying of the contents. Reagents can be pre-dispensed into wells, pads (403), or compartments formed in the tray such that they communicate individually with their corresponding FEs, but not with each other, i.e., are isolated one from another. Rather than liquid reagent, a preferred delivery approach is a dehydrated form. Dry reagent deposited at defined points on the cover communicates only with the corresponding FE fluid path when attached to the filter tray apparatus (405). Fluid retained in the FE fluid path, for example, in either the absorbent pad beneath the FEs, or the pre-filter pad over the FEs, rehydrates the dry component. It then diffuses/disperses into the FEs where it reacts with the filtered contents, i.e., to engender an indicator response.

In many cases, such as quantifying microbial content of a sample, the growth step is an essential element of the assay procedure to attain a readout result. Culture media, such as tryptic soy broth, is an exemplary reagent used to promote the replication of organisms to expand one organism into a population that can be identified, for example based on increased turbidity of the medium. In some cases, a selective media is used to preferentially grow one type of organism but not others, such as Coliscan MF which selectively grows E. coli and coliforms and serves to identify their presence in conventional assays.

For culture of organisms, the inventive method provides for incubation to be performed with the multiple-FE apparatus by exposing it to the appropriate incubation temperature that promotes the growth of the entity of interest. In the case of assaying for E. coli or fecal coliforms this is typically 35° to 45° centigrade. If the entity of assay is not an organism but does require an incubation step(s) in the analytical procedure, the multiple-FE apparatus likewise provides for that exposure condition. A preferable incubation state is as shown in FIG. 3 or 4, with the multiple-FE apparatus being covered to protect the contents from contamination and from dehydrating. The covers can be either the inlet and/or outlet chambers used in conjunction with filtration processing of the sample fluid and reagents, as shown in FIG. 3, or other discrete top covers (401) and/or bottom covers (402) as shown in FIG. 4. The incubation time that is appropriate for the particular assay depends upon the organism or entity being analyzed. As the objective is to incubate in order to develop a detectable measure of the presence of the assay entity, the time required for that to occur in the multiple-FE apparatus is preferably determined through direct testing.

With respect to the readout of results of an assay performed with the inventive method and device, in some cases the measurable indicator of the presence of the entity under analysis can simply be the entity itself. For instance, the entity may be visually identifiable in the captured material on the surface of a filter membrane based on a color or a change in appearance of the FE examined in the filter-tray apparatus. Alternatively, the presence of the entity may be determined based on an intrinsic property measurable by instrumented means.

It is common practice that the assay procedure includes indicating-means to identify the presence of the filtered entity or entities being the objective of analysis. Typical indicating-means applicable to the multi-filter element assay for microbial detection employ reporters such as: colorimetric pH indicators, fluorescent cleavage products of microbe-specific substrate-reporter conjugates, enzyme-specific markers, and antibody-reagents tagged with fluorescent indicator molecules. Similar indicating means are used to detect the specific presence of other entities, such as antigens, that can be filter assayed per the described method and device. A specific example is the fluorescent reporter 4-methylumbelliferyl-beta-D-glucuronide substrate which is used as an indicator for detecting and measuring E. coli based on their catabolism of glucuronide.

The presence of an indicator generated by the indicating-means in the multi-filter element assay is identified by interrogating each FE either visually or by analytical instrument measurements. The primary objective is to determine for each FE in the filter-tray apparatus whether the indicator is present or absent. The preferred approach to determination is an interrogation of the FE within the apparatus through its clear plastic bottom (402) and/or top cover (401) which enable the contents can be analyzed, e.g., FIG. 4. Each FE being separate, and isolated, and non-communicating with respect to fluid paths and reaction products generated by indicator-means, develops an indicator response independent of any other FE in the apparatus.

To determine the concentration of the assayed entity, the indicator presence/absence results are tallied. The number of FE that are positive, in conjunction with the information about the size or volume of each FE sample, provides a statistical basis for estimating the concentration of the entity in the tested fluid sample, e.g., microbes. The method, the basis of the multiple tube or most-probable number method, is published in Recles et al., “Most Probable Number Techniques” in “Compendium of Methods for the Microbiological Examination of Foods”, 3rd ed. 1992, at pages 105-199, and in Greenberg et al., “Standard Methods For the Examination of Water and Wastewater” 8th ed. 1992).

With multi-FE assay apparatuses that have a distribution of FE analyzing different volumes of sample, the pattern and/or distribution of positive filter elements exhibiting the indicator can be compared and contrasted against defined patterns and distributions as another approach to deriving quantitative information about the parent sample composition.

Another preferred approach to quantization of an entity using the multi-FE apparatus is to interrogate and obtain indicator presence/absence measures of the FEs at several different time points during an indicator generating response that takes time to development. An example is the assay of E. coli microorganism requiring time to grow and their presence detected on the basis of a producing a change in a fluorescent reporter associated with their metabolic activity (e.g. the 4-methylumbelliferyl-beta-D-glucuronide substrate, or measuring respiration of dissolved oxygen in the culture medium using an oxygen-sensitive indicator dye incorporated into the FE or an assay reagent). In any case, an indicator signal response develops over time in the assay condition. The amount and rate of signal change is related to the concentration of organisms, i.e., the number captured by each FE. The more entity present, the greater the change produced, and thereby, the earlier a positive identification of an indicator response can be measured/detected in the assay period, namely the presence the cells. The time-to-detection itself thus constitutes a relative measure of the concentration of the entity/organisms.

With multi-FE assays having sampled volumes that differ between sets of FEs, another advantage is gained by comparing indicator measures of one sample-size set against another. Those FE of larger sampled volume will be detectable earlier than those of less volume. The time differential between sampled-volume sets relates in a predictable manner with cell concentration, which to define the concentration are derived through reference to independently established data from calibration test standards and controls. Making multiple sets of measures of the FEs at different times in the assay period is the preferred approach in order to acquire more useful information about the time and order of appearance of detectable indicator in different sampled-volume sets.

The invention enablement for making comparisons between FEs of different sampled-volume sets provides assay-information content that constitutes internal controls which are also used to advantage. Statistical analysis of detections (FE-positive number and distribution) and of differences in detection times with different sampled volumes, permits assessment of whether the FE assay results are consistent with predicted outcomes and the assay is likely valid, or whether it does not meet expectations and should be deemed flawed and invalid.

For example, a finding that FEs of small-volume samples become positive at the same time as larger volume samples is not compliant with a statistically acceptable prediction of rank order and number. Such outcome suggests that each of the FE was overloaded with the test entity at the onset. The assay capacity was exceeded and the sample analysis needs to be repeated with a dilution to reduce the entity concentration. If the small-volume FE positive detections precede the large volume FEs, then it suggests that the test is flawed in a manner that will not benefit from merely a pre-dilution of sample.

Another example of a preferred approach to the detection of indicator-positive FEs also takes advantage of an internal control associated with the multi-FE apparatus. This is in respect to differentiating positive indicator signal from signal noise. The fundamental assay objective is to collectively determine the presence or absence of indicator in individual wells, rather than perform quantitative measures of signal in individual wells. Comparing indicator signal measures acquired from FEs of higher-volume sample against those of smaller-volume, the latter at any given point in time of the assay should exhibit less signal than the former. Signals from the smaller FEs, used as a measure of noise, thereby provide an internal control or reference for discrimination of changes in signals from other FEs as being positive responses.

There are other examples of reasons to perform more than one readout or measure of the multi-FE indicator responses over time. Obtaining an initial measure at the onset of the assay measurement phase provides reference values against which subsequent measures are compared. In some cases, the concentration of the assay entity may be so high that the sample dilution range of the multi-FE apparatus is exceed. At the point of conducting a single late stage endpoint, all FE may exhibit a positive response; therefore, a determination of the concentration of the entity is not possible. However, if multiple measures have been made starting before all FEs read positive, it is possible to extract some information about the concentration of the entity that would otherwise be lost.

With respect to controls, the inventive method and device also provides for there being included some of the FEs that are specifically dedicated to serve as positive and negative controls for the assay conditions. This includes entity standards of known concentration for internal reference purposes. For example, some FEs having “blind” fluid paths that do not allow for sample fluid to be filtered through the FEs can serve as negative controls. Such FEs that enabled, or alternatively prevented, subsequent passage of other assay reagents through the FE fluid path, provide for various quality control checks on the assay performance. Similarly, for positive controls, some FEs in the multiple-FE apparatus that have the entity of test pre-applied, likewise serve to confirm the reagents and assay conditions meet performance expectations. Such positive controls preferably include FEs that both do and do not experience exposure and filtration of the sample fluid of test. The former FEs that do contact the parent sample fluid provide useful information about interferences or inhibitors that it may contain, which bias or create errors in the assay result.

As the present invention may be embodied in several forms without departing from the spirit or essential characteristics thereof, it should also be understood that the above-described embodiments are not limited by any of the details of the foregoing description, unless otherwise specified, but rather should be construed broadly within its spirit and scope as defined in the appended claims, and therefore all changes and modifications that fall within the metes and bounds of the claims, or equivalence of such metes and bounds are therefore intended to be embraced by the appended claims.

1. A method for assaying filterable material of a fluid sample, comprising the steps of:
(i) introducing a volume of fluid test sample to the fluid-inlet side of a filtration apparatus that distributes and exposes the sample commonly to a plurality of non-communicating discrete filter elements within the filtration apparatus;
(ii) filtering a predefined volume of said fluid test sample through each said non-communicating discrete filter element thereby concentrating and/or separating filtered material to reduce said volume of respective said fluid test sample to be assayed;
(iii) removing excess residual unfiltered said fluid test sample from said filtration apparatus;
(iv) exposing each said filter element to appropriate agents or reagents to perform an analytical procedure on said filtered material; and
(v) analyzing said filter elements at one or more times for measures of said filtered material.

2. The method according to claim 1, wherein said predefined volume of said fluid test sample filtered through each filter element is the same volume for each said filter element or a different volume for each said filter element.

3. The method according to claim 1, wherein said filterable material is biological material carried in said fluid test sample.

4. The method according to claim 1, wherein said filter element provides for concentration and/or separation by physical, chemical, biological, charge and/or magnetic based methods.

5. The method according to claim 1, wherein said fluid test sample is an aqueous liquid.

6. The method according to claim 1, wherein said fluid is caused to pass through said filter apparatus by applied negative and/or positive pressure.

7. The method according to claim 1, wherein said filter apparatus and retained said filterable material after separation from said fluid test sample, is capable of being subsequently rinsed and/or exposed to other fluid or fluids, one or more times.

8. The method according to claim 1, whereby said filtration apparatus is capable of:
(i) being sealed to prevent fluid leakage, gas exchange, and/or contamination; and/or
(ii) incubated and analyzed visually or by instrumentation.

9. The method according to claim 1, wherein said agents or said reagents required to perform said analysis are administered via a component of said filtration apparatus.

10. The method according to claim 9, wherein said agents or said reagents are pre-applied to said component of said filtration apparatus in a dehydrated, liquid, or gel form.

11. The method according to claim 1, wherein said predefined volume of said fluid test sample filtered by each said filter element is controlled by an inlet-sample-holding chamber component of the filter apparatus which limits said volume of said fluid test sample presented to said filter element for filtration, and/or filtrate-collection-chamber component of said filter apparatus which limits said volume of said fluid test sample that has been exposed to said filter element.

12. A method of quantifying organisms in a fluid test sample comprising the steps of:
(i) introducing a volume of said fluid test sample to a fluid-inlet side of a filtration apparatus that distributes and exposes a sample commonly to a plurality of non-communicating discrete filter elements within said filtration apparatus;
(ii) filtering a predefined volume of said fluid test sample through each said non-communicating discrete filter elements thereby concentrating and/or separating filtered material to reduce a volume of respective said fluid test sample to be assayed;
(iii) removing excess residual unfiltered said fluid test sample from said filtration apparatus;
(iv) exposing each said filter elements to an appropriate culture media agent, growth-indicating reagent, or other analytical procedure appropriate to identify the presence of organisms of interest concentrated on said filter elements;
(v) covering said filter elements to protect them from contamination and drying;
(vi) incubating said filtration apparatus for an appropriate time and temperature to derive an indication of presence or absence of said organisms at each said filter element;
(vii) analyzing said filter elements at one or more times for measures of the presence, absence, and/or activity of filtered said organism of interest;
(viii) recording the distribution of said filter elements indicating said presence, absence, and/or activity of said organisms according to number, filtered volume, and/or position in said filtration apparatus; and
(ix) calculating the concentration of said organisms in the original said fluid test sample based on the said presence, absence, and/or activity distribution pattern.

13. A device for assaying filterable material of a fluid test sample, wherein said device comprises:
(i) a filter tray apparatus containing a plurality of non-communicating discrete filter elements each occupying and contained within a separate fluid pathway in the tray through which said fluid test sample passes providing for separation and retention of filterable material from said fluid test sample;
(ii) an inlet sample holding chamber to distribute said fluid test sample to said non-communicating discrete filter elements;
(iii) an outlet chamber to collect filtrate of said fluid test sample passed through said filter elements;
(iv) a top cover to provide a sealed enclosure protecting said filter tray apparatus; and
(v) a bottom cover to provide a sealed enclosure protecting said filter tray apparatus.

14. The device according to claim 13, wherein said filter tray apparatus provides for a pre-filter and/or a post filter or support or means for retention for each said filter element.

15. The device according to claim 13, wherein said bottom cover and/or said top cover and/or said inlet sample holding chamber and/or said outlet chamber and/or said filter tray apparatus provides for delivery of reagent to said filterable material in each said filter element.

16. The device according to claim 13, wherein said filter tray apparatus allows one or more filter elements to be used for negative and/or positive-control assay purposes.

17. The device according to claim 13, wherein said bottom cover and/or said top cover and/or said inlet sample holding chamber and/or said outlet chamber provide for enclosure of said device to prevent contamination and/or provide for negative or positive pressurization.

18. The device according to claim 13, wherein said bottom cover and/or said top cover and/or said inlet sample holding chamber and/or said outlet chamber is attachable and removable from said filter tray apparatus.

19. The device according to claim 13, wherein said inlet sample holding chamber and/or said outlet chamber provides for a vent and/or port to aspirate said fluid test sample.

20. The device according to claim 13, wherein said inlet sample holding chamber provides for wells, dividers, walls, or partitions that distribute said fluid test sample into predefined volumes for each said filter element and seal with corresponding said non-commutative filter elements.

21. The device according to claim 13, wherein said outlet chamber provides for limiting volume filtrate collection chambers contained therein to collect filtrate of predefined volume for each said filter element and seal with corresponding said non-commutative filter elements.

22. The device according to claim 21, wherein said collection chambers are made of air venting material and/or have air vents contained therein.

23. The device according to claim 21, wherein said collection chambers provide for negative or positive pressurization.

24. The device according to claim 21, wherein said collection chambers are made of a material that will not pass filtrate of said fluid test sample.

25. The device according to claim 13 that is capable of being incubated.

26. The device according to claim 13, made at least in part of plastic and/or ceramic material.

27. The device according to claim 13 having at least one portion thereof made of transmissive material capable of allowing the contents of said device to be viewed or measured by optical means.

28. The device according to claim 21, wherein said limiting volume filtrate collection chambers each provided with an air vent or a valve mechanism that stops the flow of fluid into the chamber when a specified volume has been collected.

29. The device according to claim 13, wherein said filter elements are included that provide positive and negative controls for the analytical procedure to confirm proper performance of a test.

30. The device according to claim 13, wherein said filter elements are porous filters.
